(19) United States
(12) Patent Application Publication
(10) Pub. No.: US 2020/0029882 A1
(43) Pub. Date: Jan. 30, 2020

(54) WEARABLE SENSORS WITH ERGONOMIC ASSESSMENT METRIC USAGE

(71) Applicant: Figur8, Inc., Boston, MA (US)

(72) Inventors: Nan-Wei Gong, Cambridge, MA (US); Donna Susan Scarborough, Hingham, MA (US)

(73) Assignee: Figur8, Inc., Boston, MA (US)

(21) Appl. No.: 16/593,131

(22) Filed: Oct. 4, 2019

Related U.S. Application Data
(63) Continuation of application No. 62/742,222, filed on Oct. 5, 2018.
Application No. 62/742,222 is a continuation-in-part of application No. 16/529,851, filed on Aug. 2, 2019.
Application No. 16/529,851 is a continuation-in-part of application No. 15/875,311, filed on Jan. 19, 2018.
(60) Provisional application No. 62/821,071, filed on Mar. 20, 2019.
(60) Provisional application No. 62/714,241, filed on Aug. 3, 2018.
(60) Provisional application No. 62/742,222, filed on Oct. 5, 2018.
(60) Provisional application No. 62/821,071, filed on Mar. 20, 2019.
(60) Provisional application No. 62/448,525, filed on Jan. 20, 2017.
(60) Provisional application No. 62/464,443, filed on Feb. 28, 2017.
(60) Provisional application No. 62/513,746, filed on Jun. 1, 2017.

(51) Int. Cl. A61B 5/22; A61B 5/11; A61B 5/00; A61B 5/0488

(52) U.S. Cl. CPC A61B 5/224; A61B 5/1107; A61B 5/1118; A61B 5/1128; A61B 5/0077; A61B 2562/164; A61B 5/1116; A61B 5/7278; A61B 5/6832; A61B 2562/0219; A61B 2562/06; A61B 5/0488

(57) ABSTRACT

Disclosed embodiments describe techniques for body analysis based on wearable sensors on an individual. The body analysis is based on movement assessment metric usage. The wearable sensors include muscle activity sensors, skeletal movement sensors, stretch sensors, inertial measurement sensors, or linear displacement sensors. Data is obtained from a wearable muscle activity sensor. Muscle activity over a time period is determined using the data obtained from the wearable muscle activity sensor. A movement assessment metric is calculated based on the muscle activity over the time period. The movement assessment can include body posture symmetry. The movement assessment metric is output, where the outputting can include displaying an animation of the muscle activity in a context of an overall body. The movement assessment can comprise an ergonomic assessment for the individual.

### RELATED APPLICATIONS

This application claims the benefit of U.S. provisional applications “Wearable Sensors with Ergonomic Assessment Metric Usage” Ser. No. 62/742,222, filed Oct. 5, 2018, and “Human Body Mounted Sensors with Mapping and Motion Analysis” Ser. No. 62/821,071, filed Mar. 20, 2019.

This application is also a continuation-in-part of U.S. patent application “Body Part Motion Analysis Using Kinematics” Ser. No. 16/529,851, filed Aug. 2, 2019, which claims the benefit of U.S. provisional patent application “Body Part Motion Analysis Using Kinematics” Ser. No. 62/714,241, filed Aug. 3, 2018, “Wearable Sensors with Ergonomic Assessment Metric Usage” Ser. No. 62/742,222, filed Oct. 5, 2018, and “Human Body Mounted Sensors with Mapping and Motion Analysis” Ser. No. 62/821,071, filed Mar. 20, 2019.

The patent application “Body Part Motion Analysis Using Kinematics” Ser. No. 16/529,851, filed Aug. 2, 2019 is also a continuation-in-part of U.S. patent application “Body Part Deformation Analysis Using Wearable Body Sensors” Ser. No. 15/875,311, filed Jan. 19, 2018, which claims the benefit of U.S. provisional patent applications “Body Part Deformation Analysis with Wearable Body Sensors” Ser. No. 62/448,525, filed Jan. 20, 2017, “Body Part Deformation Analysis using Wearable Body Sensors” Ser. No. 62/464,443, filed Feb. 28, 2017, and “Body Part Motion Analysis with Wearable Sensors” Ser. No. 62/513,746, filed Jun. 1, 2017.

Each of the foregoing applications is hereby incorporated by reference in its entirety.

### FIELD OF ART

This application relates generally to body analysis, and more particularly to wearable sensors with ergonomic assessment metric usage.

### BACKGROUND

The act of measuring seeks to assign a value such as a number to a feature or characteristic. The feature or characteristic relates to an event, such as time, or to an object, such as mass. A measurement may be applied not only to a static object but also to an object that is moving. The detection and measurement of motion and deformation of a given shape are paramount to a variety of computational, manufacturing, research, and other technical and professional fields. Accurate measurement of the motion or the deformation of the shape is directly pertinent to applications including medical treatment, machine vision, industrial automation, scientific biomechanics research, and three-dimensional animation, among many others. The shapes that are measured include body parts, manufactured parts, objects of interest, etc. Object differentiation can be determined using the measurements, where the object differentiation includes shape or size, constituent material, cost, or physical location, among other key parameters. When the measuring is applied to a portion of a human body, the resulting measurements have additional applications in fashion, sports, healthcare, entertainment, or gaming. Critical data, such as personal medical information, is obtained by accurate shape measurement. The medical information is routinely used to diagnose problems and to propose appropriate medical treatments. The medical treatments are critical to individual comfort, safety, and successful therapeutic outcomes.

Accurate and precise measurements of portions or all of the human body are difficult to obtain, even in a clinical setting. Consider measuring the volume in a limb of fluid buildup caused by lymphedema. This relatively simple, static, volumetric body part measurement is typically performed using a manual process, where a clinical professional uses a tape measure to make body measurements. The limb being measured is marked along a longitudinal axis using the tape measure and a marking pen. Every 1 cm or other appropriate graduation is marked off along the limb. A transverse circumference is measured and recorded at every gradation mark along the desired length of the limb. The markings are made and measurements are retaken at a subsequent clinical visit, such as one week or one month later. Total limb volume V can be approximated by assuming a step-wise linear series of cylindrical disks and summing the volumes of the linear series of cylindrical disks. Repeated use of this approximation technique can monitor lymphedema progression and/or treatment effectiveness. Note that the approximations calculated for this relatively simple example are prone to measurement inconsistencies and other opportunities for human error. A different person may be making the measurements during each patient visit. The technique of one practitioner may be better than or different from that of another practitioner, such as application of pressure during measurement. The measurement tools, such as marking pen with a tip that is several mm wide, can also contribute to inaccuracies. As a result, subtle limb shape changes, whether related to lymphedema or not, may greatly affect the accuracy of the estimated volumetric model calculation.

While making measurements of a static body part is difficult, making measurements of moving body part, such as a joint or a limb in motion, is significantly more so. The movement of a body joint occurs in three dimensions, further complicated by translation and rotation. To determine the motion of the body joint, the body joint must be moving when a measurements are taken. Body joint measurements can involve different deformations along multiple axes. To improve accuracy, multiple measurements of a repetitive motion may be required. Measurements may need to be made while the body part is under a load condition or under nominal conditions. All of these variables present an additional layer of complexity that makes measurement difficult. Further complicating the measurement tasks, the body joints are connected to other body joints, and they do not necessarily move independently of those other joints.

### SUMMARY

Body analysis is based on wearable sensors with movement assessment metric usage. The efficacy of the analysis of a body part that is in motion directly depends on accurate measurement of the motion of the body part. Analysis of body part motion has many applications including diagnoses of injuries or medical conditions, tracking the progress of healing, gauging of the efficacy of a treatment, or enhancing performance in various sports. The analysis of body part motion can also be used to identify causes of muscular fatigue or injury, and to make recommendations to increase safety, to reduce risk of injury, and to improve comfort of an individual. Techniques are disclosed for body analysis using wearable sensor. One or more sensors, including muscle activity sensors linear motion sensors, skeletal movement sensors, or inertial measurement unit (IMU) sensors, are applied to a body part of an individual. Some types of sensors, such as sensors comprising an electroactive polymer, obtain data based on changes in electrical characteristics as the sensors are stretched. Other sensors, such as the IMU, change electrical characteristics as the IMU accelerates, rotates, or changes position. The sensors are attachable to a body part such as a limb, joint, or muscle. Tape, wrap, or a garment can be applied to the body part, and the sensors can be attached to the tape, wrap, or garment using hooks, snaps, or connectors. A specialized tape such as a physical therapy tape, surgical tape, or therapeutic kinesiology tape, can be used for applying the sensors. Tape strips can be attached in various configurations to the body part. The body part can include the body, the head, a limb, and specifically across a joint, a muscle group, or other designated portion of a body part. Data including changes in electrical parameters is collected from the one or more sensors. The changes in electrical information are attributable to motion of the body part to which the one or more sensors are applied. A communication unit, coupled to the sensors, provides information to a remote computer from the sensors. The information is based on the changes in electrical characteristics such as changes in inductance, capacitance, or resistance. The communication unit can provide information using wired and wireless techniques. The remote computer receives the information provided by the communication unit. The received information is analyzed to calculate a movement assessment metric based on muscle activity over a time period.

In embodiments, a computer system for body analysis comprises: a memory which stores instructions; one or more processors attached to the memory wherein the one or more processors, when executing the instructions which are stored, are configured to: obtain data from a wearable muscle activity sensor on an individual; determine muscle activity over a time period using the data obtained from the wearable muscle activity sensor; calculate a movement assessment metric based on the muscle activity over the time period; and output the movement assessment metric. In some embodiments, a processor-implemented method for body analysis comprises: obtaining data from a wearable muscle activity sensor on an individual; determining muscle activity over a time period using the data obtained from the wearable muscle activity sensor; calculating a movement assessment metric based on the muscle activity over the time period; and outputting the movement assessment metric

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram for wearable sensors with movement assessment metric usage.

FIG. 2 is a flow diagram for outputting a movement assessment metric.

FIG. 3 is a flow diagram for body analysis.

FIG. 4 is a block diagram for motion analysis using wearable sensors.

FIG. 5A shows voluntary muscle activity during walking while wearing sneakers.

FIG. 5B shows voluntary muscle activity during walking while wearing heels.

FIG. 6 illustrates muscle activity during squats.

FIG. 7 shows a representative diagram for a wearable muscle activity sensor.

FIG. 8 illustrates detail of a capacitive sensor.

FIG. 9 shows voluntary muscle activity during pushups.

FIG. 10 shows leg sensor placements.

FIG. 11 is a system diagram for wearable sensors with movement assessment metric usage.

### DETAILED DESCRIPTION

Techniques are disclosed for body analysis using wearable sensors with movement assessment metric usage. Data is obtained from a wearable muscle activity sensor. Sensors, such as wearable muscle activity sensors, linear displacement sensors, skeletal movement sensors, or inertial measurement unit sensors, can be applied to a body part of an individual. The body part can include the body, the head, a limb, or other designated portion of a body part. The sensors can be attached to a fabric which can be attached to a body part. The fabric can include tape, a strap, a woven fabric, a knitted fabric, a garment, etc. The tape can be a specialized tape such as a physical therapy tape, surgical tape, therapeutic kinesiology tape, and so on. The attached sensors can be used to measure various parameters relating to movement of the body part. The measurement of body part can be used to perform symmetry evaluation, to evaluate a similar body part, to evaluate symmetrical operation of similar body parts, to perform micro-expression movement evaluations, to evaluate angle, force and torque of a body part, and the like. The body part can include one or more segments of the body such as the body, the head, a limb, and specifically across a joint, a muscle group, or other designated portion of a body part. The electrical characteristics of a sensor, such as a wearable muscle activity sensor or a linear displacement sensor, change as the sensor stretches. As a sensor stretches, muscle contraction or muscle activity over a time period can be determined for the body part to which the one or more sensors are attached. The electrical information can include changes in capacitance, resistance, impedance, inductance, etc. An electrical component coupled to the stretch sensor collects the changes in electrical characteristics by the one or more sensors based on motion of the body part. The electrical component collects changes in capacitance, resistance, impedance, inductance, and so on. Information from the sensors can be augmented with data obtained from other sensors such as a second sensor. A communication unit, coupled to the electrical component, provides changes in electrical information from the sensors to a remote computer. The electrical information provided by the communication unit is received by the remote computer. The remote computer is separate from the sensor, the electrical component, and the communication unit. The electrical information from the one or more sensors is analyzed to calculate a movement assessment metric. The movement assessment metric is based on muscle activity over a time period. The movement assessment metric can be output. The outputting of the movement assessment metric can be used to recommend clothing or protective gear; to recommend desk or chair usage; to analyze body posture symmetry; or to display an animation of the muscle activity of the body part.

Traditional inertial measurement unit-based systems attempt to infer the “absolute” location of a certain point of interest by integrating an acceleration reading in a 3D space. However, sampling rate issues significantly limit the accuracy of such an approach as does the accuracy of the on-board accelerometer. Drift is one of many problems that is frequently encountered by IMU-based solutions. Drift causes an error in location distance between the actual location of an object and the calculated/observed location read by the IMU. The drift error results from the accumulative error based on the calculations and worsens over time. The technique used here uses a variety of sensors to perform measurement. This approach is immune the accumulative error. Body movement such as that represented by muscle contraction output and mechanical displacement measured across a joint, can be accurately represented in a 3D space over time.

Disclosed techniques address body part analysis using wearable sensors with movement assessment metric usage. In embodiments, tape, such as physical therapy tape, therapeutic kinesiology tape, surgical tape, etc., can be applied to a body part. In other embodiments, the body part can be wrapped, placed in a garment, etc. The body part can include one or more of a limb, a joint, or a muscle. In embodiments, the tape can be applied to symmetrical body parts such as left quadricep and right quadricep, left bicep and right bicep, etc. One or more sensors can be affixed to the tape that is applied to a body part. The attaching of the one or more sensors to the tape, wrap, garment, etc., can be accomplished using hooks, a hook and loop technique, fasteners, clips, bands, connectors, and so on. The one or more sensors that can be applied can provide electrical information which, when analyzed, can be used to compute a movement assessment metric based on muscle activity over a time period.

Techniques for motion analysis can be used for body analysis. The body analysis can include tracking symmetrical body parts as the body parts are moved. The movement of the body parts can be related to tracking body part motion, body part diagnosis, body part test, body part therapy, and so on. The body part motion analysis can include acceleration and orientation information. The acceleration and orientation information relating to a body part can be collected by a six-axis or a nine-axis inertial measurement unit (IMU). The six-axis IMU can include acceleration and rotation, and the nine-axis IMU can include acceleration, rotation, and absolute direction information. A wearable muscle activity sensor or a linear displacement sensor can be used to determine motion based on stretch. The wearable muscle activity sensor or the linear displacement sensor can include an electroactive polymer.

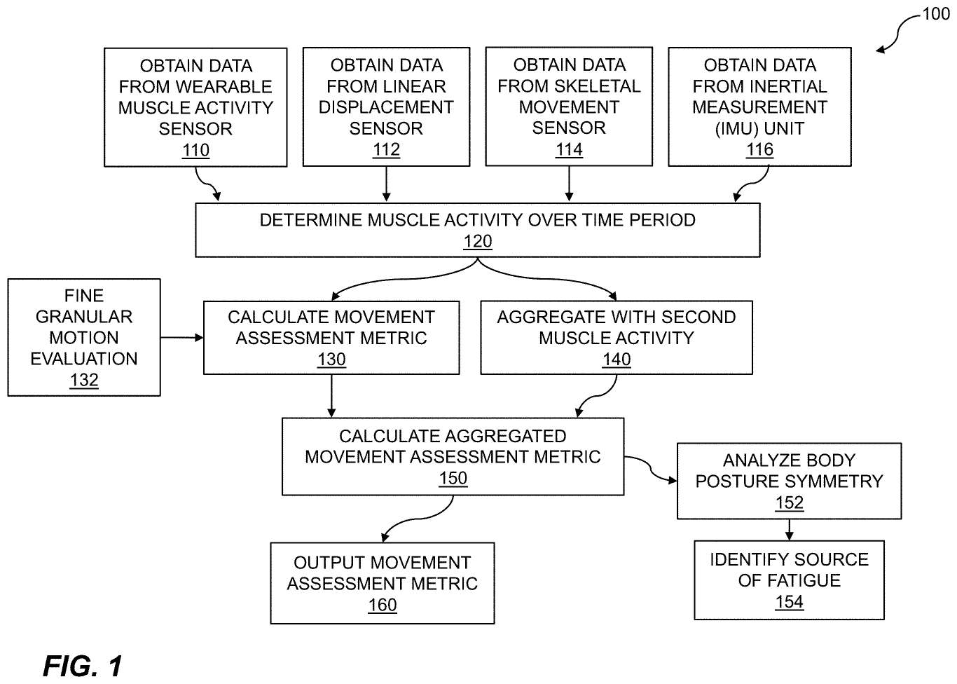

FIG. 1 is a flow diagram for wearable sensors with movement assessment metric usage. Wearable sensors are used to obtain muscle activity data from a body part of an individual, where the muscle activity data includes electrical information related to the muscle activity and or physical output of a muscle contraction. Muscle activity over a time period is determined using the data obtained from the wearable muscle activity sensor. The muscle activity that occurs over a time period is used to calculate a movement assessment metric. The movement assessment metric can include the integrity of a muscle contraction represented by timing of muscle activation or relaxation, or the magnitude of muscle contraction, or either of these metrics as represented across a joint or in unison with multiple muscle groups to qualify and quantify a body part's state of stability, joint range of motion, and the like. The movement assessment metric can be used to determine ergonomic factors such as body position, posture, or symmetry; to make recommendations to enhance sports performance; to determine medical diagnoses; to propose treatments for injuries; to propose therapies; and so on.

The movement assessment metric can relate to a specific muscle such as a bicep, triceps, quadricep, or calf muscle; a joint such as a shoulder, elbow, wrist, hip, knee, or ankle; or a group of muscles such as opposing muscles of a joint, and so on. The one or more movement assessment metrics can be used to identify stresses or strains to muscles or joints, balances or imbalances such as balances or imbalances of muscles across a joint, etc. In embodiments, one or more ergonomic assessment metrics can relate to the lumbar spine. The lumbar spine is located between the thoracic spine and the sacrum, and is commonly referred to as the “lower back”. The lumbar spine is susceptible to sprains or strains from strenuous exertion, as well as various medical conditions such as degenerative diseases of the discs between the vertebrae, aging effects such as stenosis or narrowing, and so on. A common source of pain in the lumbar spine can be attributed to spinal position or posture. An ideal posture or spinal position, one that can minimize pain and encourage health of the lumbar spine, can include a neutral spine position.

In embodiments, one or more ergonomic assessment metrics can be used for measuring a position or curvature of the spine of an individual. A comparison of the measured position of the spine of the individual to an ideal neutral spine can be determined. As discussed throughout, wearable sensors can be applied to body parts, including one or more muscles such as paraspinal muscles. Data can be collected from the one or more sensors while a person performs various tasks. The tasks can include a quasi-static task such as standing for a period of time, or various activities such as walking, running, engaging in a sport, and so on. The data collected from the one or more sensors can be used to determine muscle activity, including paraspinal muscle activity. The obtained paraspinal muscle activity can be used to calculate one or more movement assessment metrics. The movement assessment metrics are output and can be used to make various recommendations. In a usage example, data relating to the muscle activity of paraspinal muscles of a person who stands for long periods of time can be obtained and used to calculate a movement assessment metric. A movement assessment can include comparing the spinal position of the individual to the ideal neutral spinal position. Based on differences between the actual spine position and the ideal neutral spine position, recommendations can be made such as recommending clothing. The recommended clothing can include recommending footwear, where the recommended footwear can encourage a neutral position of the lumbar spine. The movement assessment metric can be used to make other recommendations such as wearing orthotic inserts in shoes or boots; placing a cushioning pad to be stood on at a workstation, and the like. In further embodiments, the one or more movement or ergonomic assessment metrics can be used for assessing maintenance of spinal neutral positions, determining safe postures to be used during high intensity, high strain, or repetitive activities, etc.

The flow 100 includes obtaining data from a wearable muscle activity sensor 110. The wearable activity sensor can be attached to tape, where the tape can include physical therapy tape or therapeutic kinesiology tape, or other tape. The wearable activity sensor can be attached to a woven material, a garment, etc. The tape, woven material, garment, and the like, can be applied to, wrapped around, or worn on a body part. Other types of sensors may be used to obtain muscle activity data. In embodiments, the muscle activity sensor includes a muscle activation sensor, where muscle activation can include producing muscle contractions. An activation sensor can be used to determine a level of muscle force. In embodiments, the muscle activity sensor can include an electromyogram (EMG) sensor. Electromyography, including electrical impedance myography (EIM), can be applied to a measurement of an intensity and a velocity of a muscle contraction event. The measurement can include electrical impedance of the muscle or muscle group. Various signals related to the muscle contraction event can be detected. In embodiments, the EMG sensor can detect the electrical current signals from activated muscles. Another example of a sensor that may be used for obtaining muscle activity data can be based on mechanical techniques. In embodiments, the muscle activity sensor can include a mechanomyogram (MMG) sensor. The MMG sensor can be used to detect movements or changes in the muscle. In embodiments the MMG sensor can detect physical deformation of a muscle.

Further wearable sensors can be used. The 100 can include obtaining data from a linear displacement sensor 112. A linear displacement sensor can be attached to tape, a wrap, a garment, and so on. The linear displacement sensor can be based on a variety of techniques. In embodiments, the linear displacement sensor can include a strip of electroactive polymer. The electroactive polymer can change electrical characteristics as it undergoes a displacement. In embodiments, the electroactive polymer is stretchable. The changes in electrical characteristics can be proportional to an amount of stretching of the electroactive polymer. The electroactive polymer can include patterns. In embodiments, the linear displacement sensor can include a conductive pattern. The pattern can be used to form electrical components, where the electrical characteristics of the electrical components change as the electroactive polymer stretches. In embodiments the conductive pattern can include an inductor formed in a substantially zigzag pattern. Other electrical components can be formed. In other embodiments, the linear displacement sensor can comprise a strip of resistive material. The flow 100 includes obtaining data from a skeletal movement sensor 114. Data from skeletal movement can be obtained using optical techniques. In embodiments, the skeletal movement sensor comprises a camera. More than one camera may be used to sense skeletal movement. The optical techniques of skeletal motion capture can be based on passive markers, active markers, time-modulated techniques, and so on. In the flow 100, the skeletal movement sensor can include a wearable inertial measurement unit (IMU) sensor 116. An IMU sensor can be used to obtain data relating to a force, an angular rate, and a position the body. In embodiments, the IMU sensor includes one or more of an accelerometer, a gyroscope, or a magnetometer.

The flow 100 includes determining muscle activity over a time period 120 using the data obtained from the wearable muscle activity sensor. Muscle activity can be related to movement of limbs or joints, which can include coordinated movements of muscles for actions such as walking, running, dancing, swimming, and so on. Muscle activity can result from interactions of forces internal to a body such as the interactions of muscles and soft tissues, with forces external to the body such as ground reaction force (GRF) and joint reaction. Muscle activity can include a contraction of a muscle, muscle deformation, a duration of a muscle contraction or deformation, and so on. Muscle activity can be determined based on other obtained data. Further embodiments can include determining the muscle activity over the time period based on the data from the linear displacement sensor. The data from the linear displacement sensor can include data related to changing electrical parameters such as inductance, capacitance, resistance, and the like. The muscle activity can be determined based on data from the skeletal movement sensor such as image data from the one or more cameras. Further embodiments can include determining the muscle activity over the time period based on the data from the IMU sensor. The data from the IMU sensor can include acceleration, rotation, or position.

The flow 100 includes calculating a movement assessment metric 130 based on the muscle activity over the time period. Movement assessment metrics can be used to gauge levels of function and abilities to perform tasks. The tasks can include walking, running, etc.; playing sports; performing work-related tasks, and so on. The movement assessment metrics can be used to perform a movement assessment of an individual. Further embodiments include evaluating the movement assessment metric based on a fine granular motion evaluation 132. The fine granular motion evaluation can be based on details relating to movement of the body part. The flow 100 further includes aggregating the muscle activity over the time period with a second muscle activity 140 over the time period. The second muscle activity can be obtained from a second sensor applied to the individual at a time that is substantially similar to the time at which the first sensor is attached to the individual. The second muscle activity may include data obtained from the individual at a different time. The first muscle activity and the second muscle activity might be compared to determine healing progress, efficacy of a treatment plan, and the like. The second muscle activity may include data obtained from other people. The data obtained from other people can be compared to the data obtained from the individual to compare muscle activity, to compare to a baseline or average, and the like.

The flow 100 includes calculating a movement assessment metric 150 based on the muscle activity over the time period. As stated throughout, the movement assessment metric can be used to gauge levels of function and abilities of an individual to perform various tasks. The tasks can include mobility tasks, other physical tasks such as sports, work-related or life-related tasks such as lifting boxes or carrying groceries, etc. Based on the movement assessment metrics, a movement assessment of an individual can be performed to determine health, fitness, ability to perform a job, and the like. In embodiments, the movement or ergonomic assessment can include analysis of body posture symmetry 152. Body posture symmetry can include raising arms, moving legs, and so on. The posture symmetry can be used to determine whether substantially similar weight is applied to each foot while standing, walking, etc. Posture symmetry can be used to determine an imbalance between left and right limbs such as arms or legs. Further embodiments include using the analysis of body posture symmetry to identify a source of fatigue 154. Fatigue can result when one limb is exerting more effort than another, such as the right arm working harder than the left arm. Further embodiments include calculating an aggregated movement assessment metric based on the muscle activity over the time period and the second muscle activity over the time period.

The flow 100 includes outputting the movement assessment metric 160. The outputting the movement assessment metric can include communicating the movement assessment metric with a remote computer. The communicating with the remote computer can include wired or wireless techniques for transferring data such as transferring data over a computer network. The remote computer can perform operations and can apply techniques for analysis and rendering of the movement assessment metric. The outputting the movement assessment metric can include displaying a point or a time of maximum stress on a muscle. In embodiments, the outputting includes a magnitude of tension on a calf muscle, a hamstring muscle, or a quadricep muscle. The outputting the movement or ergonomic assessment metric can include recommendations for changing spine-area tension or curvature. Other actions may be taken based on the outputting of the movement assessment metric. In embodiments, the outputting the movement assessment metric can include recommending clothing such as garments or footwear; recommending protective gear such as boots, gloves, pads, orthotic inserts, braces, or a helmet; etc. In further embodiments, the outputting the movement assessment metric can include recommending desk or chair usage. Various steps in the flow 100 may be changed in order, repeated, omitted, or the like without departing from the disclosed concepts. Various embodiments of the flow 100 can be included in a computer program product embodied in a non-transitory computer readable medium that includes code executable by one or more processors.

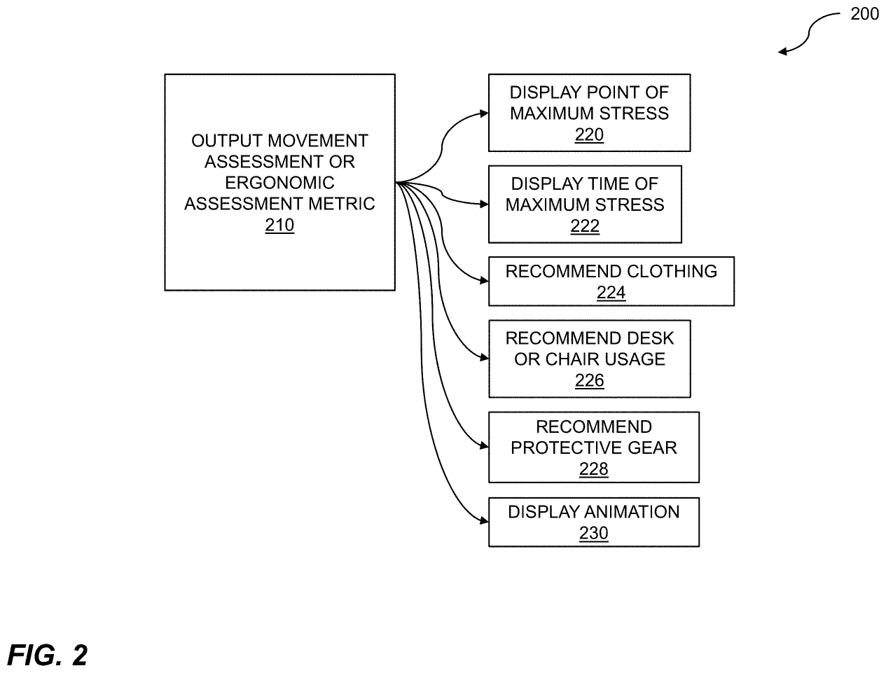

FIG. 2 is a flow diagram for outputting a movement assessment metric. As discussed throughout, a movement assessment metric or an ergonomic assessment metric can be calculated based on muscle activity over a time period. The muscle activity over a time period can be determined using data obtained from various wearable sensors such as muscle activity sensors, linear displacement sensors, skeletal movement sensors, inertial measurement unit sensors, and so on. Data can also be collected using one or more cameras. Body analysis is based on wearable sensors with movement assessment metric usage.

The flow 200 includes outputting the movement or ergonomic assessment metric 210. The movement assessment metric can include a value, a percentage, a range of values, text, and so on. The movement assessment metric can be rendered on a display such as an electronic display coupled to a smartphone or tablet, a laptop computer, a television screen, a projector, and so on. Various types of data, text, images, and so on can be rendered on the screen. The movement assessment metric can be used to highlight key data, or to make decisions such as diagnostic or treatment decisions, recommendations, and so on. The outputting the movement assessment metric can be accomplished using a variety of techniques. In embodiments, outputting of the movement assessment metric can be accomplished by a communication unit coupled to a wearable muscle activity sensor. The communication unit can communicate with a remote computer or other computing device using wired or wireless communications techniques. The remote computer can accomplish analysis, rendering, recommendations, etc.

For the flow 200, the outputting the movement or ergonomic assessment metric can include displaying a point of maximum stress 220 on a muscle. The point of maximum stress on the muscle can be rendered on a graph such as a graph of muscle contraction shown as the percentage of maximum voluntary contraction (MVC). Other data relating to the movement assessment metric can be output, rendered, and so on. For the flow 200, the outputting the movement assessment metric can include displaying a time of maximum stress 222 on a muscle. The time of maximum stress can be rendered as a chart or graph and can be rendered on a display. The time scale for the time of maximum stress on a muscle can include portions or second, seconds, etc. In embodiments, the outputting can include a magnitude of tension on a calf muscle, a hamstring muscle, or a quadricep muscle. For the flow 200, the outputting of the movement assessment metric can include recommending clothing 224. The clothing can include a wrap or strap as part of a treatment; using a brace or splint; changing shoes; and the like. In embodiments, the outputting includes a recommendation for changing spine-area tension or curvature. Changes in spine-area tension or curvature may be accomplished by changing clothing such as footwear, using appliances such as a brace or “rib belt”, adjusting posture, etc. For the flow 200, the outputting of the movement assessment metric can include recommending desk or chair usage 226. The recommendations for a desk may include adjusting a desk, using a standing desk or exercise desk, and the like. The recommendations for a chair may include adjusting a chair for correct ergonomic fit, using a balance chair such as a yoga ball chair or “core chair”, etc. For the flow 200, the outputting of the movement assessment metric can include recommending protective gear 228. The protective gear can include pads, guards, or braces; gloves or protective footwear; a helmet, and the like. For the flow 200, the outputting can include displaying an animation 230 of the muscle activity. The animation can include a representation of a body, one or more limbs, one or more muscles, and so on. In embodiments, the muscle activity can be displayed in a context of an overall body of which the muscle is a portion thereof. The animation can include movement of the body such as raising or moving arms, rotating shoulders, moving legs, moving hips, bending joints such as elbows, wrists, knees, or ankles, etc. Various steps in the flow 200 may be changed in order, repeated, omitted, or the like without departing from the disclosed concepts. Various embodiments of the flow 200 can be included in a computer program product embodied in a non-transitory computer readable medium that includes code executable by one or more processors.

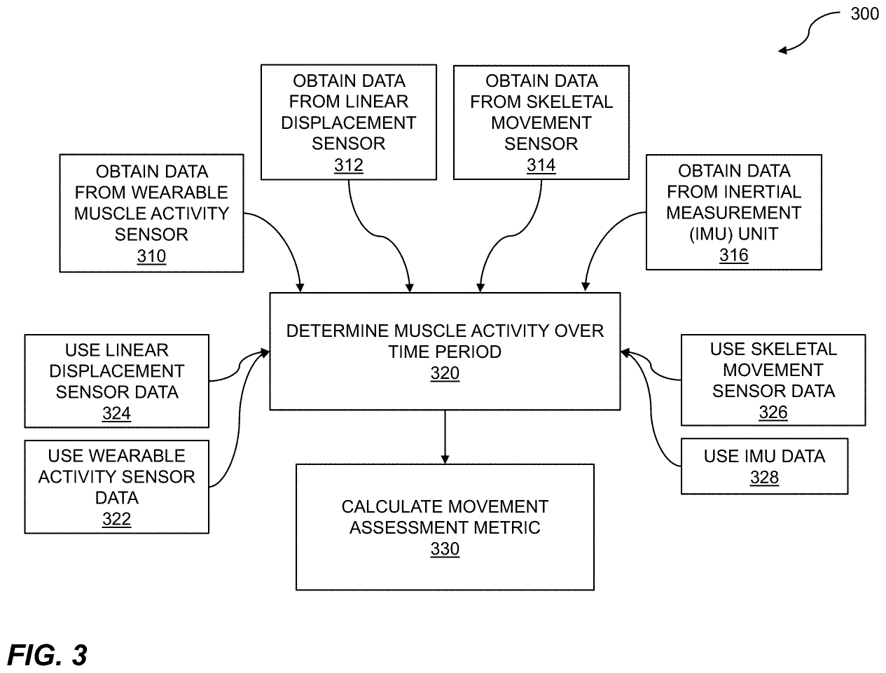

FIG. 3 is a flow diagram for body analysis. Body analysis can be performed based on wearable sensors with movement assessment metric usage. The wearable sensors can be applied to various body parts including joints or muscles. When applied to muscles, the sensors can obtain data from a wearable muscle activity sensor. Muscle activity can be determined over a time period, and a movement assessment metric can be calculated. The movement assessment metric can be output to show a point or time of maximum stress on the muscle. The movement assessment metric that is output can be used to recommend clothing or protective gear, or usage of a desk or chair.

The flow 300 includes obtaining data from a wearable muscle activity sensor 310. The wearable muscle activity sensor can be coupled to tape, fabric, a garment, etc. that can be applied to or that covers the muscle from which data is to be collected. When the muscle to which the sensor can be applied is a muscle of a limb, such as the quadricep of the left leg, then one or more sensors may be applied to a muscle of a symmetrical limb, such as the quadricep of the right leg. Data collected from corresponding limbs such as the left and right legs can be used to analyze symmetry. In embodiments, the analysis can include analysis of body posture symmetry. The flow 300 includes obtaining data from a linear displacement sensor 312. The linear displacement sensor may be applied to a body part using tape, kinesiology tape, a wrap, etc. In embodiments, the linear displacement sensor can include a strip of electroactive polymer, where the strip of electroactive polymer is stretchable. The electroactive polymer can be configured into a conductive pattern comprising various shapes such as a strip or “I” shape, a “T” shape, a “W” shape, an “X” shape, and so on. The electroactive polymer can be configured with various electrical components such as an inductor, a capacitor, a resistor, etc. In embodiments, the conductive pattern of the electroactive polymer includes an inductor formed in a substantially zigzag pattern. The electrical characteristics of the electroactive polymer can change as the electroactive polymer is stretched. In further embodiments, the linear displacement sensor can include a strip of resistive material.

In embodiments, techniques based on electrical impedance myography can be used. Electrical impedance myography (EIM) can be applied to a measurement of an intensity and a velocity of a muscle contraction event, where the measurement can include electrical impedance of the muscle or muscle group. EIM can be a noninvasive technique that can measure the electrical impedance characteristics. The electrical impedance characteristics can be used to determine health of a muscle or group of muscles, such as diagnosing a neuromuscular disease or other medical condition, assessing progression of the disease or condition, etc. The muscle health determination also can be useful for assessing the effectiveness of physical therapies, surgeries, or treatments; measuring the progress of healing; and so on.

The composition of a muscle or a group of muscles can be altered by the occurrence of disease, as can the microstructure of the muscle or group of muscles. By measuring changes in electrical impedance of the muscle or muscles using EIM, the occurrence of a disease such as a neuromuscular disease can be detected. The measurement of muscle impedance can be represented by a resistance-capacitance (RC) model, where the resistance component can be associated with cellular fluids within the muscles, and the reactance component can be associated with capacitive effects attributable to the cell membranes of the cells within the muscles. The cellular fluid can include extracellular fluid and intracellular fluid. The cell membranes can represent the capacitor dielectric separating the extracellular and intracellular fluids. Since disease can alter, at times significantly, the membranes of the cells, the impedance of the cells can also undergo significant changes. Thus, measuring the impedance of the muscle or muscle group over time can determine disease presence, disease progression, atrophy of muscle fibers, etc.

Impedance, such as electrical impedance associated with myography, is based on real components described as resistance, and imaginary components described as reactance. By applying a signal such as a sinusoidal signal to the surface of a muscle or muscle group, and by measuring the amount of time or time delay taken for the signal to pass through the muscle, a phase value can be calculated. By measuring resistance and reactance, and by calculating phase, a muscle disease may be identified. Electrical impedance myography can be impacted by physical characteristics of the patients for whom EIM is being performed. Physical characteristics of the patient can include thickness of the skin, the amount of fat under the skin (subcutaneous fat), and so on. By applying more than one sinusoidal test signal, where the sinusoidal test signals are based on different frequencies, the effects of skin and fat on impedance measurements can be reduced. Further, an amount of subcutaneous fat between the skin and the muscle may also be determined.

In embodiments, the muscle activity sensor can include an electromyogram (EMG) sensor. As discussed throughout, EMG, which can include electrical impedance myography (EIM), can be applied to a measurement of a muscle contraction event. The muscle contraction event can be characterized by an intensity and a velocity of a muscle contraction. Measurement using a sensor to obtain signal data based on EIM can include electrical impedance of the muscle or muscle group. In embodiments, the EMG sensor can detect electrical signals from nerve conduction. Another example of a sensor for obtaining muscle activity data includes mechanical techniques. In embodiments, the muscle activity sensor can include a mechanomyogram (MMG) sensor. The MMG sensor can be used to detect movements or changes in the muscle. In embodiments the MMG sensor can detect physical deformation of a muscle.

The flow 300 includes obtaining data from a skeletal movement sensor 314. Skeletal movement can be sensed using optical systems, where the optical systems can include one or more cameras. Skeletal movement can be sensed using passive markers attached to a body. The passive markers can include retroreflective material that can reflect light that can be captured by one or more cameras. Skeletal movement can be sensed using active markers, where position can be determined based on light from one or more light emitting diodes (LED) that are attached at known positions to a body part. In embodiments, the skeletal movement sensor can include a wearable inertial measurement unit (IMU) sensor 316. The IMU sensor can include one or more of an accelerometer, a gyroscope, or a magnetometer. The IMU can be used to sense movement, rotation, position, etc., of the body part to which the IMU is attached.

The flow 300 includes determining muscle activity over a time period 320. The determining muscle activity over a time period can be based on data obtained from one or more sensors, where the one or more sensors comprise various wearable sensors, cameras, and so on. In embodiments, the flow 300 includes using the data obtained from the wearable muscle activity sensor 322. The data can include electrical data, where the electrical data can include variations in electrical parameters such as inductance, capacitance, resistance, and so on. The flow 300 includes determining the muscle activity over the time period based on the data from the linear displacement sensor 324. The data from the linear displacement sensor can include values or signals relating to electrical parameters such as inductance, resistance, or capacitance, that varied as the electroactive polymer was stretched. The flow 300 includes using the data obtained from the skeletal movement sensor 326. The data from the skeletal movement sensor can include optical data, passive marker data, active marker data, and so on. The flow 300 includes determining the muscle activity over the time period based on the data from the inertial measurement (IMU) sensor 328. The data from the IMU can include signals or data from components of the IMU such as signals or data from the accelerometer, the gyroscope, or the magnetometer.

The flow 300 includes calculating a movement assessment metric 330. The movement assessment metric can be based on the data collected from the various sensors, cameras, etc., that can be used for body analysis. The calculating the movement assessment metric can be based on the muscle activity over the time period, on linear displacement, on skeletal movement, on acceleration, rotation, or position, and so on. Further embodiments include aggregating the muscle activity over the time period with a second muscle activity over the time period. The second muscle activity can include muscle activity of another limb of the person; muscle activity from other points in time such as when the muscle was injured and when it has healed; muscle activity of other people, and so on. The second muscle activity can be from another sensor on the same muscle. In embodiments, the second muscle activity can be in a similar direction to the muscle activity. Multiple sensors may be used on a large muscle such as a quadricep, on multiple muscles in a limb, etc. In other embodiments, the second muscle activity can be in a substantially perpendicular direction to the muscle activity. Various steps in the flow 300 may be changed in order, repeated, omitted, or the like without departing from the disclosed concepts. Various embodiments of the flow 300 can be included in a computer program product embodied in a non-transitory computer readable medium that includes code executable by one or more processors.

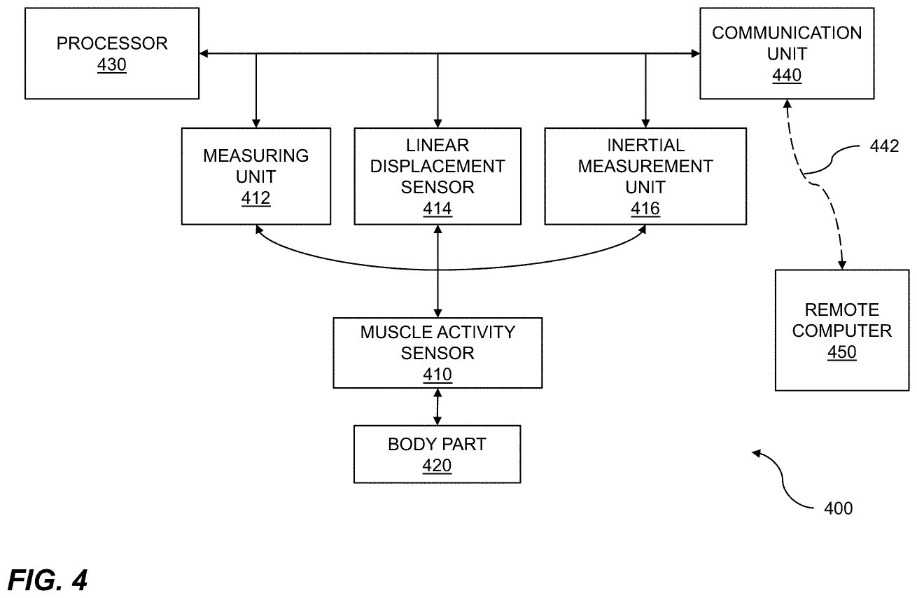

FIG. 4 is a block diagram for motion analysis using wearable sensors. The motion analysis can support wearable sensors with movement assessment usage. Body analysis is based on wearable sensors. The wearable sensors can include muscle activity sensors, linear displacement sensors, skeletal movement sensors, inertial measurement unit (IMU) sensors, and the like. A wearable sensor, such as a muscle activity sensor, changes electrical characteristics as it displaces. Displacement can include bending, stretching, and so on. Data is obtained from a wearable muscle activity sensor, and muscle activity over a time period is determined. A movement assessment metric is calculated based on the muscle activity over the time period, and the movement assessment metric is output.

A block diagram for motion analysis using wearable sensors 400 is shown. The block diagram 400 includes a muscle activity sensor 410. Tape (not shown) can be attached to a body part 420 from which data is to be obtained. A muscle activity sensor 410 can be attached to the tape. The muscle activity sensor can be attached to the tape using connectors, hooks, snaps, Velcro™, etc. In some embodiments, the muscle activity sensor includes a capacitive, resistive, or inductive sensor, where the electrical characteristics of the sensor change as the sensor is bent, stretched, and so on. The tape attached to the body part can include physical therapy tape or therapeutic kinesiology tape, a woven material, a garment or wrap, etc. The body part 420 to which the stretch sensor is attached can include one or more of a joint such as a knee, shoulder, elbow, wrist, ankle, spine, and so on. The body part can include a muscle such as a bicep, triceps, quadricep, trapezius, deltoid, etc. The muscle activity sensor can be coupled to a wearable measuring sensor unit 412. The wearable sensor unit 412 can include two or more body sensors. The wearable sensor can collect electrical information including capacitance, resistance, impedance, inductance, and so on. The muscle activity sensor can be coupled to linear displacement sensor 414. The linear displacement sensor can include a strip or other shape such as a T, X, or W shape, where the strip or shape includes a stretchable electroactive polymer. The linear displacement sensor can include a conductive pattern such as a zigzag pattern. The electrical characteristics of the electroactive polymer change as the polymer is pulled or stretched. The muscle activity sensor can be coupled to an inertial measurement unit (IMU) 416. The inertial measurement unit can capture movement information, attitude information, position information, etc. The wearable measuring unit 412 can be coupled to a processor 430. The processor 430 can be used for controlling the one or more wearable sensors, for generating electrical signals to operate the wearable sensors, for collecting data from the wearable sensors, for analyzing data from the wearable sensors, and so on. The measuring unit can be coupled to a communication unit 440. The communication unit 440 can provide wired and/or wireless communications 442 between the muscle activity sensor 410, measuring unit 412, the linear displacement sensor 414, and/or the inertial measurement unit 416, and a remote computer 450. The communication unit 440 can include Ethernet™, Bluetooth™, Wi-Fi, Zigbee™, infrared (IR), near field communication (NFC), and other communications capabilities. The communication unit 440 can send information including movement, attitude, position, percent maximum voluntary contraction, and so on.

In other embodiments, the sensor configuration can include a bend sensor. One or more bend sensors can be applied to a body part of an individual and can be used for body part motion analysis. The one or more bend sensors can be used to measure motion of the body part with one or more degrees of freedom. Various techniques can be used to implement a bend sensor such as correlating the bend sensor to a compliant capacitive strain sensor. A compliant capacitive strain sensor can comprise a dielectric layer sandwiched between two conducting electrode layers. The dielectric layer and the electrode layers can be can use flexible materials, where the flexible materials can include polymers. The flexible materials such as the polymers can include natural rubber, silicone, acrylic, and so on. Since the polymers can typically be insulators, the electrodes of the bend sensor can be formed by introducing conducting particles into the polymers, where the conducting particles can include nickel, carbon black, and the like. In order for the capacitive strain sensor to be applied to the body part, one or more compliant capacitive strain sensors or other strain sensors can be affixed to a material such as tape that can be applied to the body part, a fabric that can enwrap the body part, a garment that can be worn on the body part, and so on.

The compliant capacitive strain sensor can measure strain based on the amount of displacement experienced by the strain sensor. The ability of a compliant capacitive strain sensor to measure strain can be limited by the amount of displacement that can be sustained by the strain sensor before the strain sensor is temporarily or permanently damaged. Excessive strain applied to the strain sensor can cause electrical parameters of the strain sensor, such as the resistance of the strain sensor, to change significantly. The significant change in resistance of the strain sensor can include an “open circuit” (high resistance) resulting from a damaged or destroyed strain sensor.

An application of a sensor, such as the configuration shown, to a body part (e.g. a shoulder) can be used to determine angle measurements for the shoulder. In embodiments, angle measurements can include sagittal plane flexion and extension. In addition to angle measurements for a given body part, muscle function assessment can also be performed. In embodiments, muscle function assessment can include displacement of muscle contraction that can occur during an activity. The activity can include normal physical activity such as yoga and strenuous physical activity such as swimming, rowing, rock climbing, and so on. Peak displacement of a muscle can be based on maximum contraction of key superficial muscle groups. A sensor can be attached to a targeted muscle group, over the location of greatest muscle mass displacement. In addition to peak muscle displacement for muscle function determination, an amount of time required to reach peak muscle contraction can be recorded. Other sensors can be applied to shoulder measurements. In embodiments, the inertial measurement unit (IMU) can be used to track acceleration and orientation of a body part such as a shoulder. Based on measurements collected from the IMU, intersegmental movement can provide information on movement patterns across anatomical joints. The information based on the intersegmental movement provides information on a fluidity of movement and a quality of motion. This information can provide side to side comparison of movement of the anatomical joints for healthy populations in contrast with injured populations.

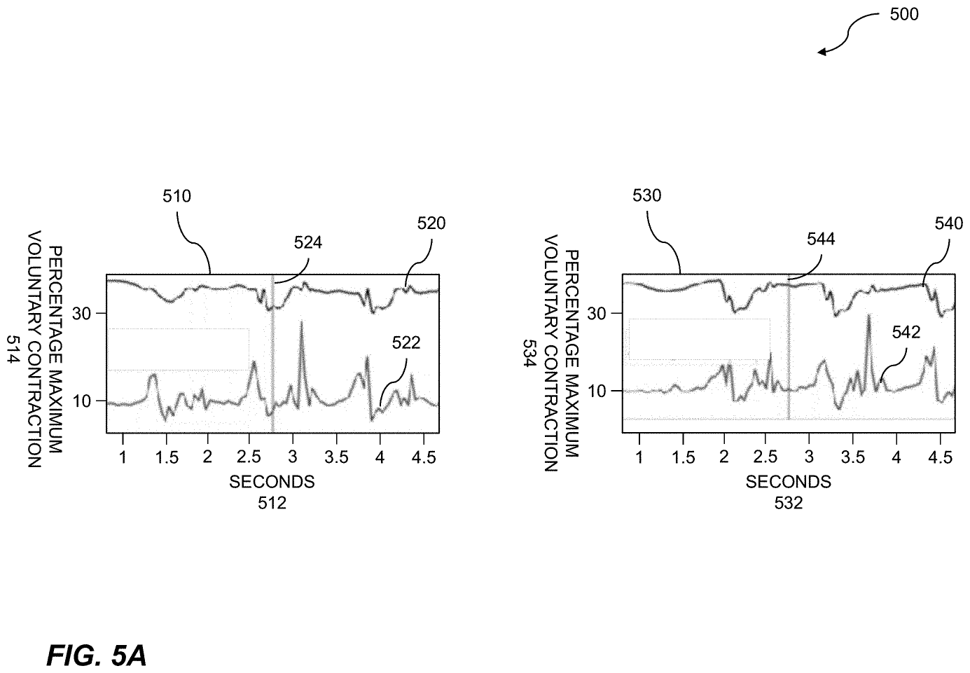

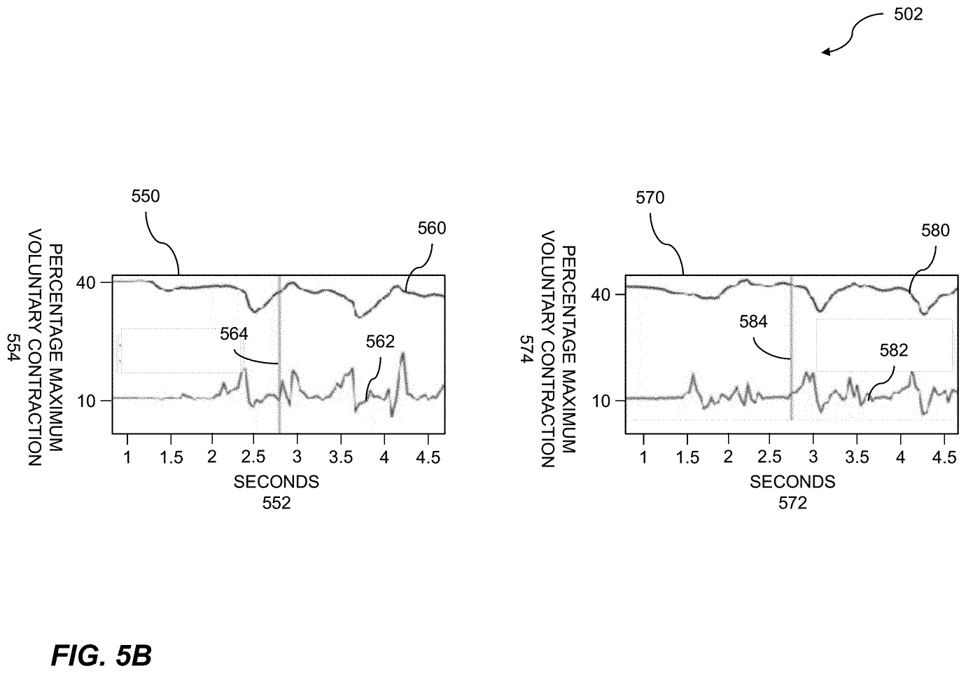

FIG. 5A shows voluntary muscle activity during walking while wearing sneakers. As described throughout, sensors such as wearable sensors can be attached to various body parts of a person, to monitor muscle activity. The sensors can include wearable sensors with movement assessment metric usage. The person to whom the sensors are attached may perform various actions such as walking, running, dancing, swimming, and the like. Data is obtained from a wearable muscle activity sensor. Muscle activity over a time period is determined using the data obtained from the wearable muscle activity sensor. A movement assessment metric is calculated based on the muscle activity over the time period. The movement assessment metric is output, where the outputting can include displaying data points or an animation, recommending clothing or protective gear, recommending desk or chair usage, and the like.

One or more wearable sensors can be applied to a person who is walking. In the illustration 500, the person is walking while wearing sneakers. Sensors can be applied to calf muscles to determine calf muscle activity as the person walks. The wearable sensors can be applied to one leg or two legs, pelvis and trunk, or to other body portions. As discussed throughout, the one or more wearable sensors can include muscle activity sensors, linear displacement sensors, skeletal movement sensors, inertial measurement unit (IMU) sensors, and the like. Data can be obtained from the sensors and the data can be plotted. Plot 510 shows percent maximum voluntary contraction 514 over time 512 for the left leg. The plot shows data relating to magnitude acceleration 520 and left calf muscle contraction 522. At a point in time 524, values for acceleration and contraction can be determined. Plot 530 shows percent maximum voluntary contraction 534 over time 532 for the right leg. The plot shows data relating to magnitude acceleration 540 and right calf muscle contraction 542. At a point in time 544, a value for acceleration and a value for contraction can be determined. Other output data can be plotted. In embodiments, the outputting includes a magnitude of tension on a calf muscle, a hamstring muscle, or a quadricep muscle. Data output on a quadricep muscle is described elsewhere. Recall that a movement assessment metric can be calculated based on the data obtained from the one or more wearable sensors. In embodiments, the movement assessment can include analysis of body posture symmetry. The body posture symmetry can include assessment of left calf muscle contraction to right calf muscle contraction. Further embodiments include using the analysis of body posture symmetry to identify a source of fatigue.

FIG. 5B shows muscle activity during walking while wearing heels 502. A person can wear various types of footwear such as fashion or lifestyle footwear including sandals, loafers, flats, or heels; footwear for specific activities such as hiking boots or bicycling shoes; and so on. Wearable sensors can be applied to the person, and data can be obtained that relates to percentage maximum voluntary contraction over time. The data can relate to a muscle of a left limb, a corresponding muscle of a right limb, and so on. The data collected from the muscle of the left limb and the data collected from the muscle of the right limb can be compared for analysis of body posture symmetry, to identify a source of fatigue or potential injury, and so on.

One or more wearable sensors can be applied to a person who is walking while wearing heels 502. Sensors can be applied to calf muscles or other leg muscles to determine calf muscle activity or other muscle activity as the person walks in the heels. The wearable sensors can be applied to one leg or two legs. The one or more wearable sensors can include various cameras or sensors such as muscle activity sensors, linear displacement sensors, skeletal movement sensors, inertial measurement unit (IMU) sensors, etc. The data that is collected or obtained can be plotted. Plot 550 shows left leg percentage maximum voluntary contraction 554 over time 552. The plot shows data relating to magnitude acceleration 560 and left calf muscle contraction 562. At a point in time 564, values for acceleration and contraction can be determined. Plot 570 shows right leg percentage maximum voluntary contraction 574 over time 572. The plot shows data relating to magnitude acceleration 580 and right calf muscle contraction 582. At a point in time 584, a value for acceleration and a value for contraction can be determined. Data obtained for the left leg, shown in plot 550, can be compared to data obtained for the right leg, shown in plot 570, for symmetry and other analysis. Further, data obtained for a person walking in heels can be compared to data obtained for the person walking in sneakers. As a result of outputting a movement assessment metric, a change in footwear may be recommended. In embodiments, techniques are used to measure alignment of the spine in its natural posture. The sensors can be placed on paraspinal muscles and curvature of the spine be measured to quantify change resulting from ergonomic alterations such as shoe wear, clothing and or activity. This quantification will help in posture stability and ergonomic analysis.

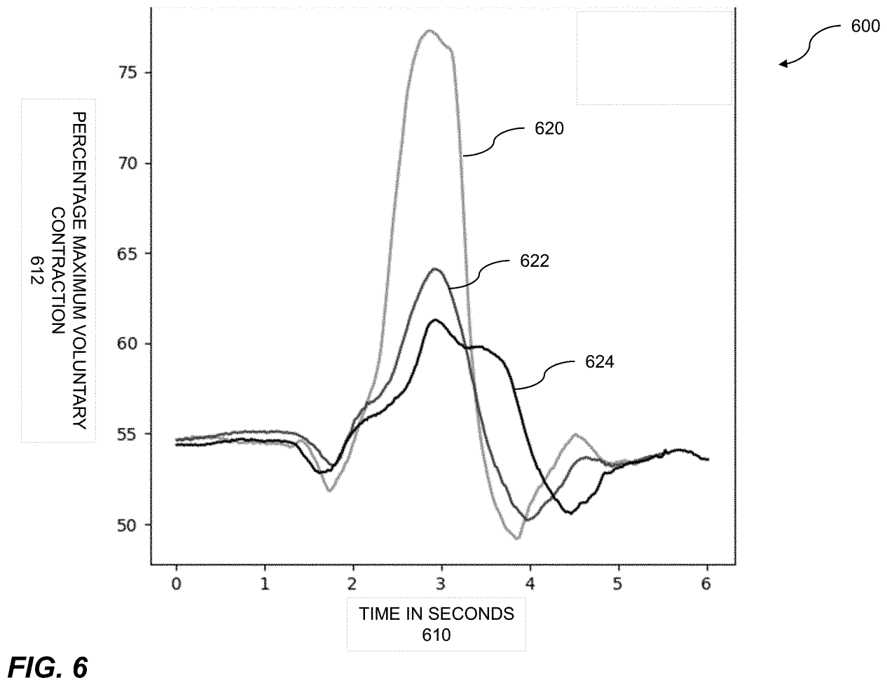

FIG. 6 illustrates muscle activity during squats. Sensors can be attached to various body parts of a person as that person performs one or more repetitions, or reps, of an exercise. Among the many exercises that can be performed, a squat is an exercise that can engage muscles of the lower body. Wearable sensors can be applied to muscles of the lower body to collect data relating to muscle activity of the lower body. The wearable sensors can include wearable sensors with movement assessment metric usage. In embodiments, data is obtained from a wearable muscle activity sensor, and muscle activity is determined over a time period. A movement assessment metric is calculated, and the movement assessment metric is output.

One or more wearable sensors can be applied to a person who is performing squats. The sensors can be applied to various body parts such as to one leg or two legs. If the sensors are applied to two legs, then percentage maximum voluntary contraction can be compared between the two legs for determining body posture symmetry, muscle activity symmetry, and the like. The person to whom the wearable sensors have been applied can perform squats, and data can be collected from the one or more wearable sensors. The person can perform the squats in bare feet or can perform the squats while wearing various types of footwear. In the plot 600, percentage maximum voluntary contraction 612 is plotted over time 610. In the plot, data from sensors was collected from the person while in bare feet 620, while wearing sneakers 622, and while wearing high heels 624. The various plots indicated that the percentage maximum voluntary contraction is significantly higher while the person is wearing high heels than when the person is wearing sneakers or is in bare feet. In embodiments, analysis of body posture symmetry can be used to identify a source of fatigue. The body posture symmetry may be also be used to determine or assess a cause of pain such as muscular pain in the left leg or the right leg. In embodiments, the movement assessment metric can include recommending clothing. The clothing can include shoes, among other items of clothing.

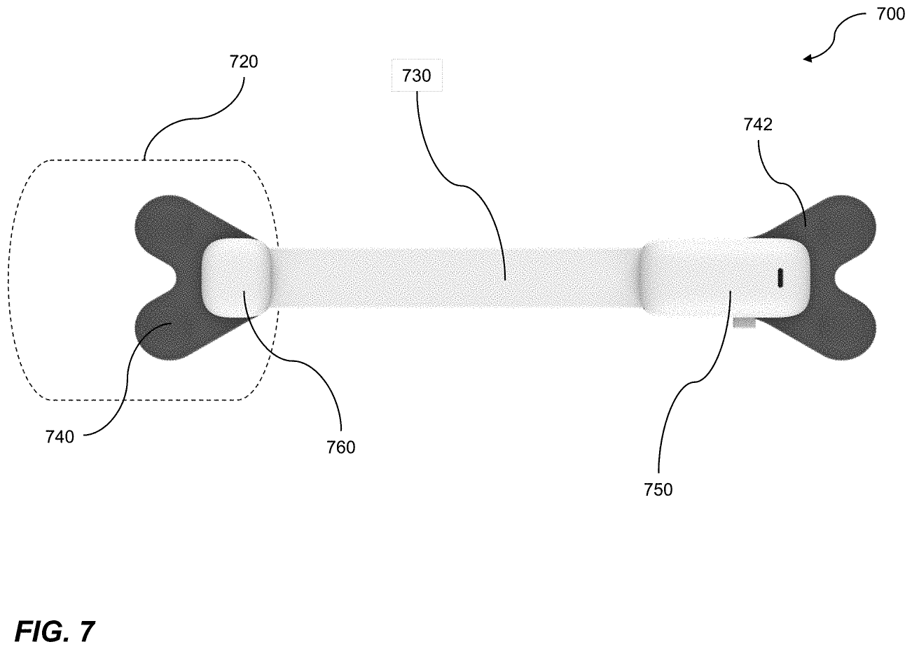

FIG. 7 shows a representative diagram of a wearable muscle activity sensor 700. Wearable sensors based on muscle activity sensors, skeletal motion sensors, wearable inertial measurement unit (IMU) sensors, stretch sensors, or linear displacement sensors, are used for body analysis. A sensor, such as a stretch sensor or an IMU, changes electrical characteristics as it stretches or moves, respectively. The electrical characteristics can include resistance, capacitance, inductance, reluctance, and so on. The stretching of the stretch sensor can correspond to movement of a body part to which the sensor is attached. Similarly, motion of an IMU can include acceleration, rotation, or position of the body part. A collector or sensor coupled to the stretch sensor to collect changes in electrical characteristics based on motion of the body part. A communication unit provides information from the sensor or collector to a receiving unit. Data is obtained from a wearable muscle activity sensor. Muscle activity is determined over a time period using the data obtained from the wearable muscle activity sensor. A movement assessment metric is calculated based on the muscle activity over the time period. The movement assessment metric is output. A communication unit can provide the movement assessment metric that is output to a receiving unit. The information that is received is displayed, where the display can include an animation of the body part. The body part can be displayed in a context of an overall body.

A representative diagram of a wearable muscle activity sensor is shown. Tape 720 such as physical therapy tape or therapeutic kinesiology tape can be attached to a body part. The body part can include one or more of a knee, shoulder, elbow, wrist, hand, finger, thumb, ankle, foot, toe, hip, torso, spine, arm, leg, neck, jaw, head, back, and so on. One or more stretch sensors can be included. A stretch sensor 730 can be attached to tape 720 using an anchors 740. Anchor 742 can be attached to another piece of tape (not shown) or another device. The anchors can include hooks. While an “end-to-end” arrangement of stretch sensors is shown, other arrangements can include a t-shape, an x-shape, a spider-shape, w-shape, and so on. A cover 750 or 760 can cover an electrical component. The electrical component can include a power supply, a communication unit, an electrical characteristic processing unit, an IMU, etc. In embodiments, sensors or tape can be applied to lumbar spine, thoracolumbar spine, or thoracocervical locations and movement or ergonomic metrics tracked for neutral spine postures during variation in tasks or clothing, such as shoes wear

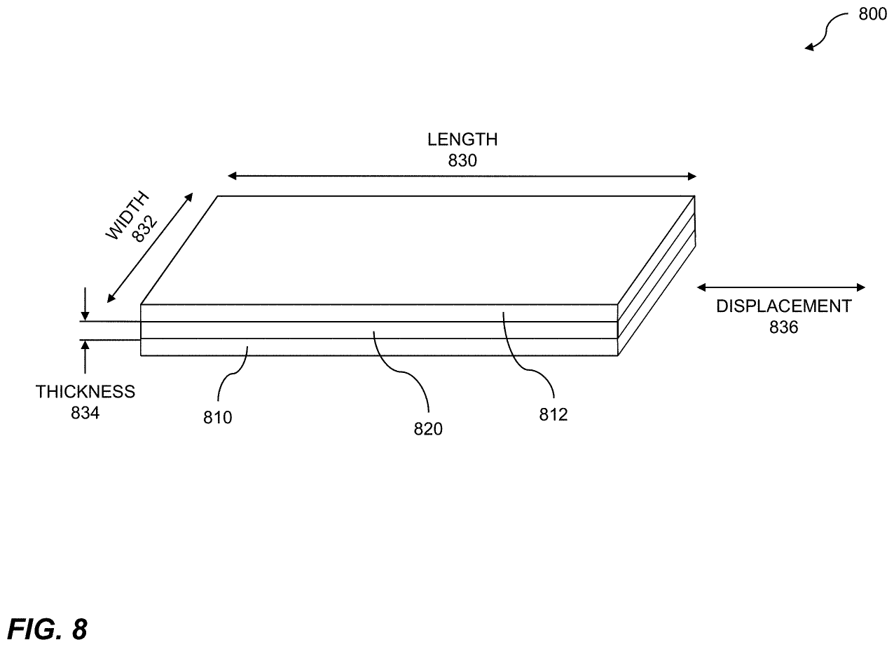

FIG. 8 illustrates detail of a capacitive sensor. A capacitive sensor can be included among various wearable sensors, where the wearable sensors can include wearable sensors with movement assessment metric usage. Data can be obtained from a wearable muscle activity sensor, and muscle activity can be determined over a time period using the data obtained from the wearable muscle activity sensor. A movement assessment metric can be calculated based on the muscle activity over the time period, and the movement assessment metric can be output.

Illustration 800 shows a three-dimensional view of a capacitive sensor implementation. The capacitive sensor has a length 830 and a width 832. Embedded between conductive layers 810 and 812 is a dielectric material 820 with thickness 834. The conductive layers 810 and 812 can be attached to a fabric (not shown). The fabric may be a tape such as a therapeutic kinesiology tape, among other such tapes. Therapeutic kinesiology tape often exhibits properties of readily allowing deformation or stretching along only one axis. In this illustration, the length 830 deforms easily, but the width 832 does not readily deform. As the sensor is deformed or stretched along the length 830, a displacement 836 is indicated. However, it is clear that the aforesaid stretching will affect the dielectric material 820 and cause it to become thinner. When one dimension of a three-dimensional solid material with finite volume is expanded, another dimension must contract to maintain the constant, finite volume. The thinning of dielectric material 820 will result in increased capacitance between the conductive layers 810 and 812. The capacitance may be approximated using the general parallel plate capacitor equation C=K*Eo*A/d, where Eo is the permittivity of free space (8.85410⁻¹²), K is the dielectric constant of the material, A is the overlapping surface area of the plates, d is the distance between the plates, and C is capacitance.

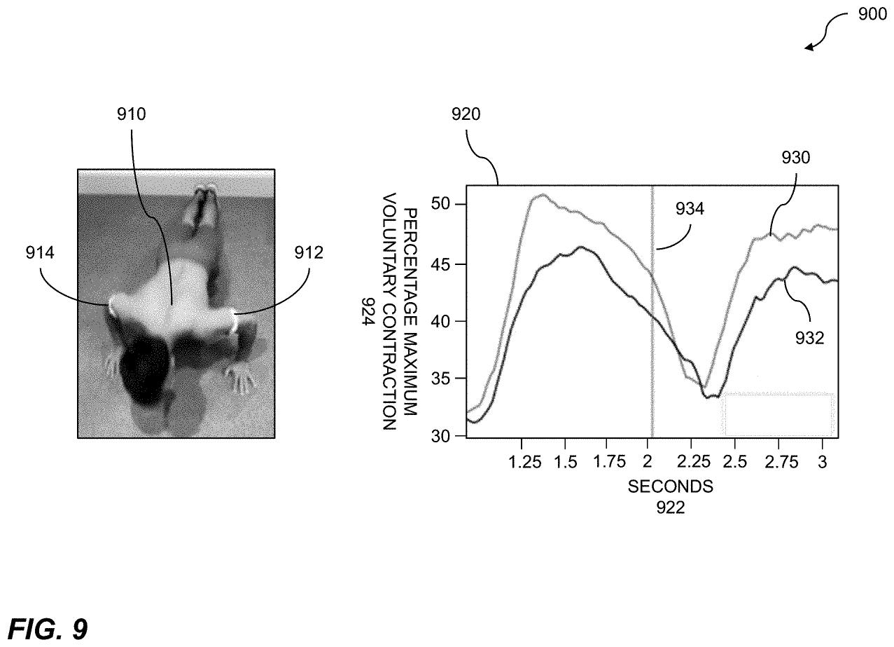

FIG. 9 shows voluntary muscle activity during pushups. As described throughout, body analysis can be performed by determining voluntary muscle activity. The voluntary muscle activity can be determined over time based on a wearable sensor with movement assessment metric usage. Data can be obtained from a wearable muscle activity sensor. Muscle activity can be determined over a time period using the data obtained from the wearable muscle activity sensor. A movement assessment metric can be calculated based on the muscle activity over the time period, and the movement assessment metric can be output.

Muscle activity during pushups 900 is shown. An individual 910 can perform a variety of exercises such as pushups or other activities such as walking, running, swimming, cycling, dancing, and so on. Further activities can include standing, sitting, balancing, and the like. One or more wearable sensors can be applied to the individual, such as sensor 912 on the individual's left arm, and sensor 914 on the individual's right arm. The sensor or sensors can be placed so as to monitor muscle activity of one or more muscles such as biceps, triceps, and the like. The wearable sensors can include muscle activity sensors, skeletal movement sensors, inertial measurement unit sensors, stretch sensors, and so on. Data obtained from the sensors can be used to determine muscle activity over time. The muscle activity can be plotted.

A plot 920 of percentage maximum voluntary contraction 924 over time 922 in seconds is shown. The plot includes data 932 collected from the sensor 912 on the left arm, and data 930 collected from the sensor 914 on the right arm. At a point in time 934, the percentage maximum voluntary contraction of the right arm can be compared to that of the left. The percentage maximum voluntary contraction of the right sensor is seen to be greater than that of the left. The difference in voluntary muscle contractions between the two arms may be attributable to a variety of factors such as the person performing the pushups being right handed, asymmetry in the muscles of the left arm and the right arm, injury, and so on. The differences between the percentage maximum voluntary contraction between the left and right arms can cause fatigue or pain in the right arm. By identifying the differences between the two arms, recommendations with respect to posture, stance, position, motion, etc. can be made. As time passes, the differences in magnitude between the plots of the data from the right arm and the data of the left arm can decrease or converge. The decrease in the difference of the magnitudes can indicate states of the muscles such as muscle fatigue. The higher frequency variations shown in the plots of the maximum voluntary contraction can further indicate fatiguing of the muscles of the person performing the pushups. Such high frequency variations shown in the plots may present visual as shaking or jittering of the arms as the person continues to perform pushups.

As mentioned throughout, the one or more movement assessment metrics that can be computed based on data obtained from one or more wearable sensors can be output. The outputting can include a magnitude of tension in a muscle such as a calf, hamstring, or quadricep muscle; a changing spine-area tension or curvature; and so on. The movement assessment metrics can be analyzed. In embodiments, the movement or ergonomic assessment can include analysis of body posture symmetry. The body posture symmetry can be analyzed to identify a source of fatigue, a potential cause of injury, the effectiveness of a treatment plan including physical therapy, and so on. Symmetry may further include other analyses to determine muscle balance across a joint. In embodiments, the analysis can include analyzing muscle output across the joint. The muscle output can provide stability for postural control, where postural control can include a ratio of output provided by muscles such as quadriceps relative to an antagonist muscle group, such as the hamstrings. When the muscles work in balance, or are in symmetry, then the muscles perform their tasks efficiently while reducing the risk of injury. When the muscles are in imbalanced across a joint, then joints such as the knees, shoulders, spine, and so on, are at risk of injury. In embodiments, assessing maintenance of spine neutral postures can be used to indicate idealistic and safe postures during high strain or during repetitive activities for both ergonomics purposes and competitive sports activities.

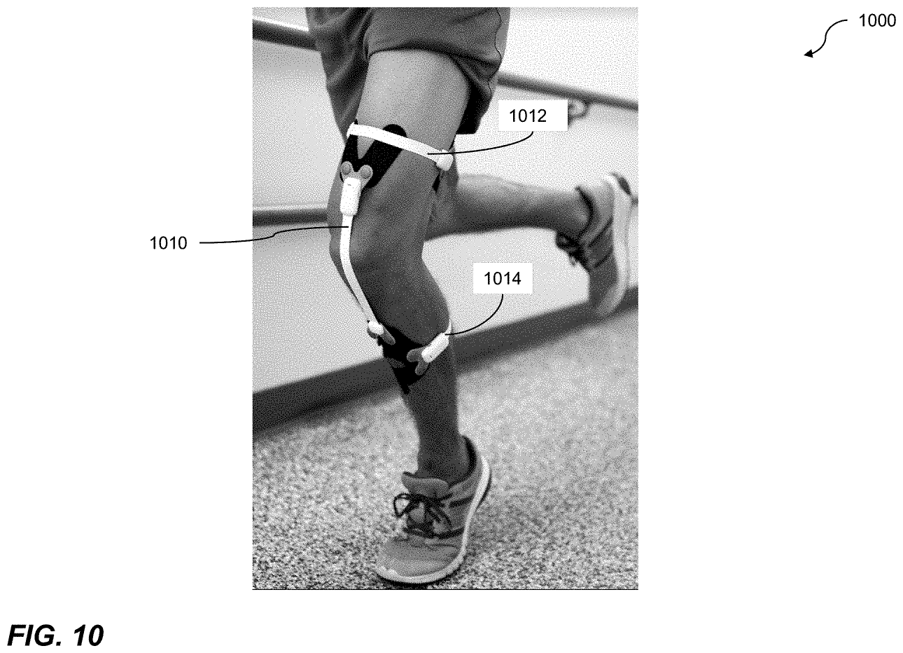

FIG. 10 shows leg sensor placements. One or more sensors such as wearable sensors can be applied to body parts, where the body parts can include a muscle, a limb, a joint, and so on. Sensors may also be applied to a corresponding muscle, limb, or joint to support analysis such as analysis of body posture symmetry. The body posture symmetry can be used to identify asymmetries between muscle activity or joint motion of limbs such as left and right arms or legs; joints such as left and right shoulders, knees, or wrists, and so on. The body posture symmetry can be used to identify a source of fatigue, a root cause of joint or muscle injury or pain, the effect on muscle activity or joint motion of footwear, clothing, protective gear, and the like. The sensors that are applied to the body parts can include wearable sensors with movement assessment metric usage. Data obtained from a wearable muscle activity sensor is used to determine muscle activity over a time period. A movement assessment metric is calculated, and the movement assessment metric is output.

The placement of wearable sensors on a leg is shown 1000. Three wearable sensors are shown applied to the leg. In further embodiments, other numbers of sensors may be applied to the leg. The wearable sensors can include muscle activity sensors, skeletal movement sensors, linear displacement sensors, inertial measurement unit sensors, and so on. The sensors can be applied to the leg in order to measure muscle or joint activity, displacement, deformation, etc. A first sensor 1010 can be applied to the knee. The sensor can be used to measure motion of the knee such as a number of degrees of flexion of the knee as the person to whom the sensor is applied engages in various activities such as standing, walking, running, bicycling, dancing, swimming, and so on. Other sensors may be applied to the leg. A second sensor is applied to the quadricep 1012. The second sensor can be a muscle activity sensor, linear displacement sensor, and IMU sensor, etc. One or more other sensors can be applied to the leg. A third sensor 1014 can be applied to the calf. The third sensor can be a sensor of similar type to the first or second sensor, or may be dissimilar to one or more other sensors. The data collected or obtained from the sensors may be aggregated. While sensors applied to one leg are shown, one or more further sensors can be attached to the right leg. When sensors are applied to both legs, or other limbs, then the data collected from the sensors of the left limb and the sensors of the right limb can be analyzed for symmetry such as body posture symmetry. The data obtained from the sensors may also be used to quantify differences in muscle activity, joint movement, etc. The quantified difference can be correlated with changes in footwear, protective gear, clothing, and so on.

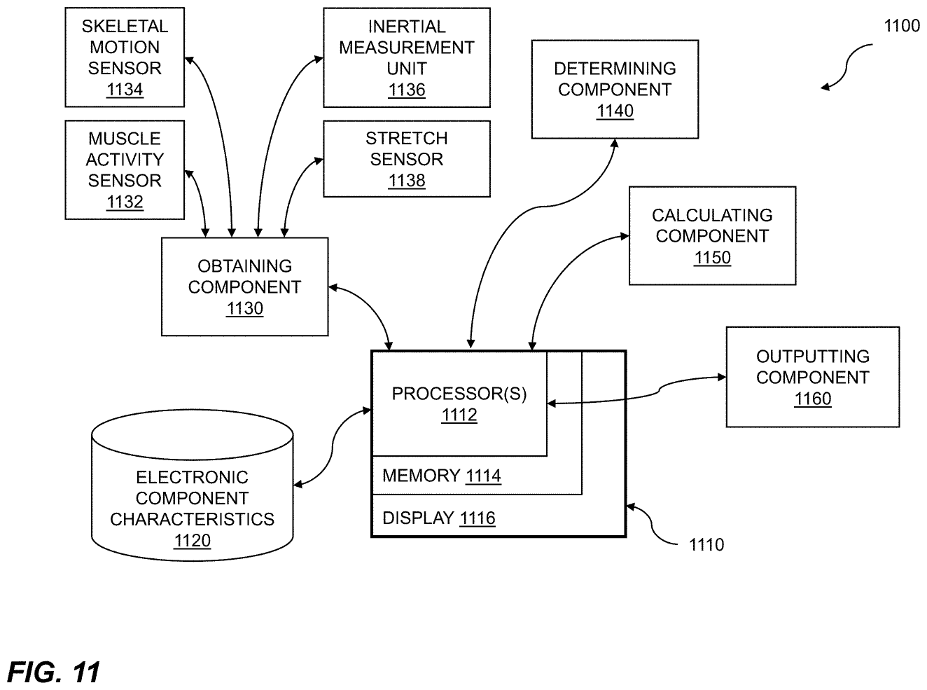

FIG. 11 is a system diagram for wearable sensors with movement assessment metric usage. Movement assessment metrics can be used to describe a motion of a body part. The body part motion can include a muscle action such as the action of a bicep, tricep, quadricep, etc.; motion of a body part including a joint(s) such as a lumbothoracic region, shoulder, elbow, hip, knee, ankle, wrist; and so on. Sensors, including body-attachable or wearable sensors, can be used to analyze motion or action of a body part. Various types of sensors can be applied to a body part of an individual, where the application can be accomplished using hooks to attach to tape or straps, suction cups, wraps, garments, safety equipment, and so on. The sensors can include muscle activity sensors, skeletal movement sensors, stretch sensors, or inertial measurement units (IMU). The IMUs can include an accelerometer, a gyroscope, a magnetometer, etc. The electrical characteristics of the stretch sensor or the IMU can change based on the sensor stretching, accelerating, moving, and the like. The electrical characteristics can include inductance, resistance, or capacitance, where the electrical characteristics change based on movement of the body part. Data is obtained from a wearable muscle activity sensor, and muscle activity is determined over a time period using the data obtained from the wearable muscle activity sensor. A movement assessment metric is calculated based on the muscle activity over the time period, and the movement assessment metric is output. In embodiments, the outputting includes displaying an animation of the muscle activity, where the muscle activity is displayed in a context of an overall body.

The system 1100 can include an analysis computer 1110. The analysis computer can include one or more electronic components which can be used to analyze electrical information from wearable sensors. The analysis performed by the analysis computer can generate a movement assessment metric. The analysis computer 1110 can comprise one or more processors 1112, a memory 1114 coupled to the one or more processors 1112, and a display 1116. The display 1116 can be configured and disposed to present collected data, analysis, intermediate analysis steps, instructions, algorithms, or heuristics, and so on. In embodiments, one or more processors are attached to the memory, where the one or more processors, when executing the instructions which are stored, are configured to: obtain data from a wearable muscle activity sensor on an individual; determine muscle activity over a time period using the data obtained from the wearable muscle activity sensor; calculate a movement assessment metric based on the muscle activity over the time period; and output the movement assessment metric.

The system 1100 can include an electronic component characteristics component 1120. The electronic component characteristics can include a library of lookup tables, inductance characteristics, resistance characteristics, capacitance characteristics, functions, algorithms, routines, code segments, and so on, that can be used for the analysis of the electrical information collected from sensors such as the wearable muscle activity sensor. In a usage example, the electrical component characteristics can include a lookup table that enables mapping of an electrical signal from a stretch sensor to millimeters of motion of the body part. The system 1100 can include an obtaining component 1130. The obtaining component can act as an interface between one or more sensors and the analysis computer 1110. The obtaining component can obtain data from one or more wearable sensors, where the data can include electrical signals. The electrical signals can be generated by the wearable muscle activity sensor 1132, a skeletal movement sensor 1134, an inertial measurement unit 1136, a stretch sensor 1138, and so on.

The system 1100 can include a determining component 1140. The determining component 1140 can determine muscle activity over a time period using the data obtained from the wearable muscle activity sensor or other sensor. The determining component can include electronic components or other hardware for determining inductance, resistance, or capacitance. The determining can include determining current, voltage, resistance, capacitance, impedance, and/or inductance. A generating component (not shown) can include hardware for generating direct current and/or alternating current signals used for obtaining resistance and/or capacitance measurements. Typically, the current values are low (e.g. microamperes) and in embodiments, the frequency range includes signals from about 100 hertz to about 1 megahertz.

The system 1100 can include a calculating component 1150. The calculating component can calculate a movement assessment metric based on the muscle activity over the time period. The calculating can be based on data such as electrical data relating to the muscle activity that can be collected from the various sensors including wearable sensors. The wearable sensors can include muscle activity sensors, skeletal movement sensors, inertial measurement units, stretch sensors, acceleration sensors, rotational motion sensors, magnetic field sensors, etc. The system 1100 can include an outputting component 1160. The outputting component can output the movement assessment metric. The movement assessment metric can include various metrics such as displaying a time of maximum stress on a muscle. The time of maximum stress on a muscle can include the time of a takeoff, landing, and other motion. The movement assessment metric can include recommendations, such as recommending clothing or protective gear, recommending furniture usage such as using a desk or a chair, suggesting that the person take a break from activity, and so on. The movement assessment can be used for analysis, such as analyzing body posture symmetry, analyzing a source of muscle fatigue, and the like. In embodiments, the outputting can include displaying an animation of the muscle activity, where the muscle activity can be displayed in a context of an overall body.

The system 1100 can include a computer program product embodied in a non-transitory computer readable medium for body analysis, the computer program product comprising code which causes one or more processors to perform operations of: obtaining data from a wearable muscle activity sensor; determining muscle activity over a time period using the data obtained from the wearable muscle activity sensor; calculating a movement assessment metric based on the muscle activity over the time period; and outputting the movement assessment metric.

Each of the above methods may be executed on one or more processors on one or more computer systems. Embodiments may include various forms of distributed computing, client/server computing, and cloud-based computing. Further, it will be understood that the depicted steps or boxes contained in this disclosure's flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. The elements and combinations of elements in the block diagrams and flow diagrams, show functions, steps, or groups of steps of the methods, apparatus, systems, computer program products and/or computer-implemented methods. Any and all such functions—generally referred to herein as a “circuit,” “module,” or “system”—may be implemented by computer program instructions, by special-purpose hardware-based computer systems, by combinations of special purpose hardware and computer instructions, by combinations of general purpose hardware and computer instructions, and so on.

A programmable apparatus which executes any of the above-mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are neither limited to conventional computer applications nor the programmable apparatus that run them. To illustrate: the embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM), an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the causal entity.

While the invention has been disclosed in connection with preferred embodiments shown and described in detail, various modifications and improvements thereon will become apparent to those skilled in the art. Accordingly, the foregoing examples should not limit the spirit and scope of the present invention; rather it should be understood in the broadest sense allowable by law.

What is claimed is:

1. A processor-implemented method for body analysis comprising:
obtaining data from a wearable muscle activity sensor on an individual;
determining muscle activity over a time period using the data obtained from the wearable muscle activity sensor;
calculating a movement assessment metric based on the muscle activity over the time period; and
outputting the movement assessment metric.

2. The method of claim 1 further comprising obtaining data from a movement sensor on the individual and further calculating the movement assessment metric based on the individual's movement.

3. The method of claim 2 wherein the movement sensor comprises a wearable inertial measurement unit (IMU) sensor.

4. The method of claim 3 further comprising determining the muscle activity over the time period based on the data from the IMU sensor.

5. (canceled)

6. The method of claim 2 wherein the movement sensor includes a camera.

7. The method of claim 1 further comprising obtaining data from a linear displacement sensor.

8. The method of claim 7 further comprising determining the muscle activity over the time period based on the data from the linear displacement sensor.

9. The method of claim 7 wherein the linear displacement sensor comprises a strip of electroactive polymer.

10. The method of claim 9 wherein the electroactive polymer is stretchable.

11-13. (canceled)

14. The method of claim 1 wherein the muscle activity sensor includes a muscle activation sensor.

15. The method of claim 1 wherein the muscle activity sensor comprises an electromyogram (EMG) sensor.

16. (canceled)

17. The method of claim 1 wherein the muscle activity sensor comprises a mechanomyogram (MMG) sensor.

18. (canceled)

19. The method of claim 1 further comprising aggregating the muscle activity over the time period with a second muscle activity over the time period.

20. The method of claim 19 further comprising calculating an aggregated movement assessment metric based on the muscle activity over the time period and the second muscle activity over the time period.

21. The method of claim 19 wherein the second muscle activity is in a similar direction to the muscle activity.

22. The method of claim 19 wherein the second muscle activity is in a substantially perpendicular direction to the muscle activity.

23. The method of claim 1 wherein the outputting the movement assessment metric comprises displaying a point of maximum stress on a muscle.

24. The method of claim 1 wherein the outputting the movement assessment metric comprises displaying a time of maximum stress on a muscle.

25-26. (canceled)

27. The method of claim 1 wherein the movement assessment includes analysis of body posture symmetry.

28-32. (canceled)

33. The method of claim 1 wherein the outputting comprises displaying an animation of the muscle activity.

34. The method of claim 33 wherein the muscle activity is displayed in a context of an overall body of which the muscle is a portion thereof.

35. The method of claim 1 further comprising evaluating the movement assessment metric based on a fine granular motion evaluation.

36. The method of claim 1 wherein the movement assessment metric comprises an ergonomic assessment.

37. A computer system for body analysis comprising:
a memory which stores instructions;
one or more processors attached to the memory wherein the one or more processors, when executing the instructions which are stored, are configured to:
obtain data from a wearable muscle activity sensor on an individual;
determine muscle activity over a time period using the data obtained from the wearable muscle activity sensor;
calculate a movement assessment metric based on the muscle activity over the time period; and
output the movement assessment metric.

38. A computer program product embodied in a non-transitory computer readable medium for body analysis, the computer program product comprising code which causes one or more processors to perform operations of:
obtaining data from a wearable muscle activity sensor on an individual;
determining muscle activity over a time period using the data obtained from the wearable muscle activity sensor;
calculating an movement assessment metric based on the muscle activity over the time period; and
outputting the movement assessment metric.
